(19) United States
(12) Patent Application Publication
(10) Pub. No.: US 2018/0318013 A1
(43) Pub. Date: Nov. 8, 2018

(54) System and Method for Determining Ablation Parameters

(71) Applicant: St. Jude Medical, Cardiology Division, Inc., St. Paul, MN (US)

(72) Inventors: Jan O. Mangual-Soto, Rho (IT); Pier Alessandro Giorgetti, Milano (IT); Michele Ciani, Roma (IT); Sergio De Mura, Firenze (IT)

(21) Appl. No.: 15/968,347

(22) Filed: May 1, 2018

Related U.S. Application Data
(60) Provisional application No. 62/501,357, filed on May 4, 2017.

(51) Int. Cl. A61B 34/10; A61B 18/00; A61B 90/00

(52) U.S. Cl. CPC A61B 34/10; A61B 18/00; A61B 90/37; A61B 2034/107; A61B 2017/00292; A61B 2018/00702; A61B 2018/00761; A61B 2034/105; A61B 2034/102; A61B 2018/00577

(57) ABSTRACT

Parameters for cardiac ablation can be determined using a map of one or more biological properties of a tissue to be ablated, such as tissue thickness. The biological properties are used to compute a transmurality index map. In turn, the transmurality index map can be used to determine one or more of ablation energy level, ablation time, and ablation contact force to achieve a transmural lesion. Graphical representations of the biological property maps, the transmural index map, and/or the ablation parameters can be output, for example on geometric models of the heart and/or the ablation catheter.

### CROSS-REFERENCE TO RELATED APPLICATIONS

This application claims the benefit of U.S. provisional application No. 62/501,357, filed 4 May 2017, which is hereby incorporated by reference in its entirety as though fully set forth herein.

### BACKGROUND

The present disclosure relates generally to cardiac therapeutic procedures, such as cardiac ablation. In particular, the present disclosure relates to systems, apparatuses, and methods for determining ablation parameters, such as ablation energy level (e.g., power, voltage, and/or current), ablation time, and ablation contact force, suitable for the creation of a transmural lesion. As used herein, the term “transmural lesion” means a lesion that extends from the endocardial surface to the epicardial surface, with low voltage amplitude across the thickness of the myocardium.

It is known that contact force (e.g., how hard an ablation catheter is pressing into the tissue), time (e.g., for how long ablation energy is applied to the tissue), and energy level (e.g., the power, voltage, and/or current of the ablation energy applied to the tissue) are variables that contribute to the creation of a transmural lesion. For example, U.S. Pat. Nos. 8,641,705 and 9,149,327, which are hereby incorporated by reference as though fully set forth herein, describe a lesion size index (“LSI”) that is a function of ablation contact force, ablation time, and ablation current. By manipulating these interdependent variables to achieve a sufficiently high LSI, a practitioner can increase the likelihood of creating a transmural lesion. As another example, U.S. provisional application No. 62/331,398, which is also incorporated by reference as though fully set forth herein, also describes an LSI that is a function of ablation contact force, ablation time, and ablation current.

The LSI described in the foregoing patents and patent applications is of particular use in the treatment of atrial arrhythmias by ablation. Atrial tissue thickness is typically subtle, ranging from about 1 mm to about 2.5 mm, with little variability.

Ventricular tissue, however, is typically thicker than atrial tissue. The thickness of ventricular tissue is also more variable than that of atrial tissue. Thus, the LSI described in the foregoing patents and applications is not as well-suited to the treatment of ventricular arrhythmias by ablation.

### BRIEF SUMMARY

Disclosed herein is a method of determining parameters for cardiac ablation, including the following steps: receiving a tissue biological property map for a cardiac region to be ablated; computing a transmurality index map using the tissue biological property map; and determining one or more of ablation energy level, ablation time, and ablation contact force to achieve a transmural lesion using the computed transmurality index map. The method can also include outputting a graphical representation of the tissue biological property map.

According to aspects of the disclosure, the tissue biological property map includes a tissue thickness map. If desired, an iconographic indication of local tissue thickness can be output on a geometric model of an ablation catheter.

The method can also include outputting a graphical representation of the transmurality index map. For example, the graphical representation of the transmurality index map can be output on a geometric model of the cardiac region to be ablated. Alternatively or additionally, the graphical representation of the transmurality index map can be output as a bullseye plot.

In additional aspects of the disclosure, one or more of ablation energy level, ablation time, and ablation contact force can be output graphically. For example, a numerical value for the one or more of ablation energy level, ablation time, and ablation contact force can be displayed on a geometric model of the cardiac region to be ablated.

The tissue biological property map for a cardiac region to be ablated can be received by: receiving a segmented model of the cardiac region to be ablated; and determining the tissue biological property map from the segmented model.

The step of determining one or more of ablation energy level, ablation time, and ablation contact force to achieve a transmural lesion using the computed transmurality index map can include, given values for two of ablation energy level, ablation time, and ablation contact force, determining a remaining one of ablation energy level, ablation time, and ablation contact force from the computed transmurality index map.

Also disclosed herein is a method of performing cardiac ablation, including: computing a transmurality index map using tissue thickness information for a cardiac region to be ablated; determining one or more of ablation energy level, ablation time, and ablation contact force to achieve a transmural lesion within the cardiac region to be ablated from the transmurality index map; and delivering ablation energy to the cardiac region to be ablated according to the determined one or more of ablation energy level, ablation time, and ablation contact force.

According to aspects of the disclosure, the step of computing a transmurality index map using tissue thickness information for a cardiac region to be ablated can include computing a transmurality index map using tissue thickness information derived from a segmented model of the cardiac region to be ablated.

In additional aspects of the disclosure, the method further includes outputting a graphical representation of the transmurality map on at least one of a bullseye plot and a geometric model of the cardiac region to be ablated.

In still further aspects of the disclosure, the step of determining one or more of ablation energy level, ablation time, and ablation contact force to achieve a transmural lesion within the cardiac region to be ablated from the transmurality index map includes, given values for two of ablation energy level, ablation time, and ablation contact force, determining a remaining one of ablation energy level, ablation time, and ablation contact force using the transmurality index map.

The method can also include graphically outputting the one or more of ablation energy level, ablation time, and ablation contact force, such as by displaying a numerical value for the one or more of ablation energy level, ablation time, and ablation contact force on a geometric model of the cardiac region to be ablated. It is also contemplated that the tissue thickness information for the cardiac region to be ablated can be output as iconography on a geometric model of an ablation catheter.

The instant disclosure also provides a cardiac ablation control system, including: an ablation parameter determination processor configured to: receive as input a tissue thickness map for a cardiac region to be ablated; compute a transmurality index map using the tissue thickness map; and determine one or more ablation parameters selected from the group consisting of ablation energy level, ablation time, and ablation contact force to achieve a transmural lesion in the cardiac region to be ablated from the computed transmurality index map.

Optionally, the ablation parameter determination processor can be further configured to: receive as input values for two ablation parameters; and compute a value for a remaining ablation parameters from the values received as input for the two ablation parameters and the computed transmurality index map. It can also be further configured to graphically output the determined one or more ablation parameters.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic diagram of an exemplary electroanatomical mapping system.

FIG. 2 depicts an exemplary catheter that can be used in connection with aspects of the instant disclosure.

FIG. 3 is a schematic diagram of a contact ablation system in accordance with embodiments disclosed herein.

FIGS. 4A through 4F are graphical representations of data used according to aspects of the instant disclosure.

FIG. 5 is a graphical representation of the correlation between the lesion width and lesion depth parameters according to aspects disclosed herein.

FIG. 6 is a flowchart of representative steps that can be followed according to exemplary embodiments disclosed herein.

FIG. 7 is an exemplary graphical representation of tissue thickness or transmurality index in greyscale on a three-dimensional model of a left ventricle.

FIG. 8 is an exemplary bullseye plot of transmurality index for a left ventricle.

FIG. 9 shows representative plots of ablation power (upper plot) and ablation contract force (lower plot) versus transmurality index.

FIG. 10 is an exemplary graphical representation of optimal ablation time, given ablation energy level and ablation contact force, output in greyscale on a three-dimensional model of a left ventricle.

FIG. 11 depicts the graphical output of a value, such as a local tissue thickness, a transmurality index, or an ablation parameter, as iconography in connection with a graphical representation of an ablation catheter.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

The instant disclosure provides systems, apparatuses, and methods for determining ablation parameters suitable for the creation of a transmural lesion. For purposes of illustration, aspects of the disclosure will be described in connection with ventricular mapping and ablation. It should be understood, however, that the teachings herein can be applied to good advantage in other contexts, including, without limitation, atrial mapping and ablation.

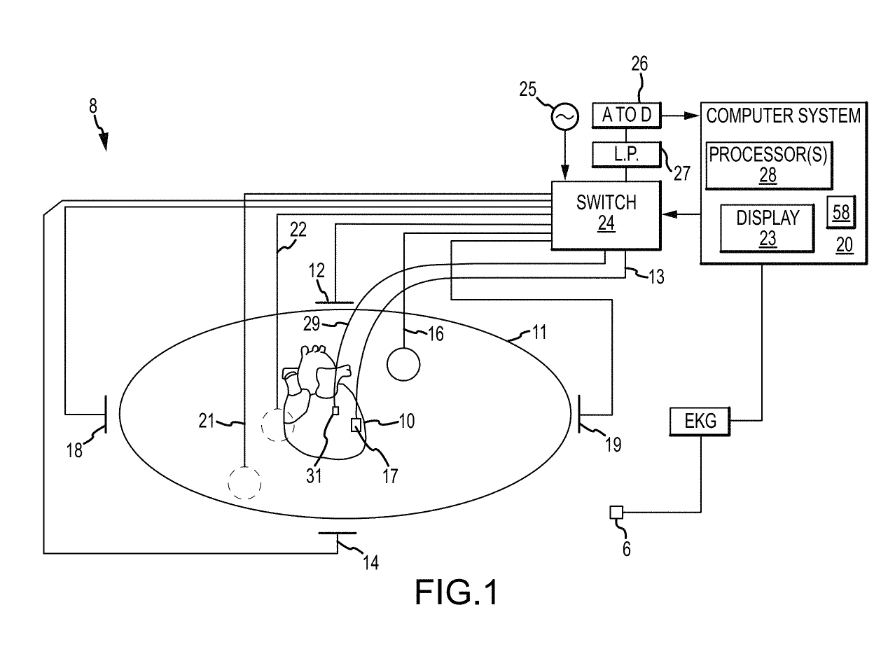

FIG. 1 shows a schematic diagram of an exemplary electroanatomical mapping system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data at a plurality of points along a cardiac surface and store the measured data in association with location information for each measurement point at which the electrophysiology data was measured, for example to create a diagnostic data map of the patient's heart 10. In some embodiments, and as discussed further herein, the system 8 can determine transmurality indices and/or ablation parameters.

As one of ordinary skill in the art will recognize, and as will be further described below, system 8 determines the location, and in some aspects the orientation, of objects, typically within a three-dimensional space, and expresses those locations as position information determined relative to at least one reference.

For simplicity of illustration, the patient 11 is depicted schematically as an oval. In the embodiment shown in FIG. 1, three sets of surface electrodes (e.g., patch electrodes) are shown applied to a surface of the patient 11, defining three generally orthogonal axes, referred to herein as an x-axis, a y-axis, and a z-axis. In other embodiments the electrodes could be positioned in other arrangements, for example multiple electrodes on a particular body surface. As a further alternative, the electrodes do not need to be on the body surface, but could be positioned internally to the body.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 16, 22 are applied along a third axis generally orthogonal to both the x-axis and the y-axis, such as along the sternum and spine of the patient in the thorax region, and may be referred to as the Chest and Back electrodes. The heart 10 lies between these pairs of surface electrodes 12/14, 18/19, and 16/22.

An additional surface reference electrode (e.g., a “belly patch”) 21 provides a reference and/or ground electrode for the system 8. The belly patch electrode 21 may be an alternative to a fixed intra-cardiac electrode 31, described in further detail below. It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only a single lead 6 and its connection to computer 20 is illustrated in FIG. 1.

A representative catheter 13 having at least one electrode 17 is also shown. This representative catheter electrode 17 is referred to as the “roving electrode,” “moving electrode,” or “measurement electrode” throughout the specification. Typically, multiple electrodes 17 on catheter 13, or on multiple such catheters, will be used. In one embodiment, for example, the system 8 may comprise sixty-four electrodes on twelve catheters disposed within the heart and/or vasculature of the patient. Of course, this embodiment is merely exemplary, and any number of electrodes and catheters may be used.

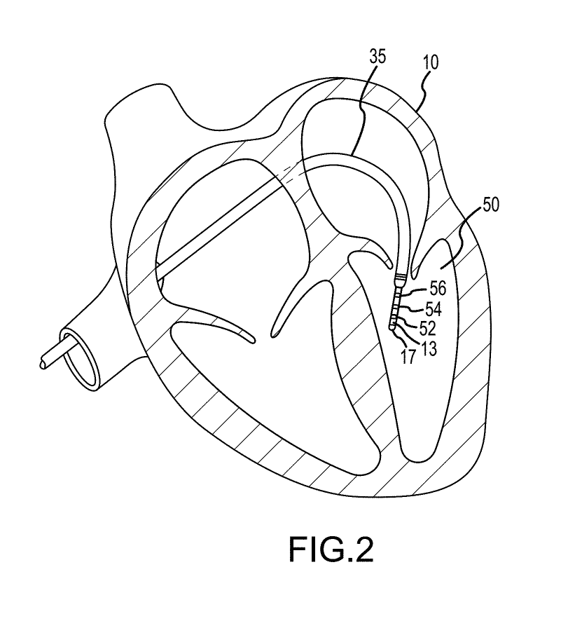

Likewise, it should be understood that catheter 13 (or multiple such catheters) are typically introduced into the heart and/or vasculature of the patient via one or more introducers and using familiar procedures. For purposes of this disclosure, a segment of an exemplary multi-electrode catheter 13 is shown in FIG. 2. In FIG. 2, catheter 13 extends into the left ventricle 50 of the patient's heart 10 through a transseptal sheath 35. The use of a transseptal approach to the left ventricle is well known and will be familiar to those of ordinary skill in the art, and need not be further described herein. Of course, catheter 13 can also be introduced into the heart 10 in any other suitable manner.

Catheter 13 includes electrode 17 on its distal tip, as well as a plurality of additional measurement electrodes 52, 54, 56 spaced along its length in the illustrated embodiment. Typically, the spacing between adjacent electrodes will be known, though it should be understood that the electrodes may not be evenly spaced along catheter 13 or of equal size to each other. Since each of these electrodes 17, 52, 54, 56 lies within the patient, location data may be collected simultaneously for each of the electrodes by system 8.

Similarly, each of electrodes 17, 52, 54, and 56 can be used to gather electrophysiological data from the cardiac surface. The ordinarily skilled artisan will be familiar with various modalities for the acquisition and processing of electrophysiology data points (including, for example, both contact and non-contact electrophysiological mapping), such that further discussion thereof is not necessary to the understanding of the techniques disclosed herein. Likewise, various techniques familiar in the art can be used to generate a graphical representation from the plurality of electrophysiology data points. Insofar as the ordinarily skilled artisan will appreciate how to create electrophysiology maps from electrophysiology data points, the aspects thereof will only be described herein to the extent necessary to understand the instant disclosure.

Returning now to FIG. 1, in some embodiments, an optional fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20 may comprise, for example, a conventional general-purpose computer, a special-purpose computer, a distributed computer, or any other type of computer. The computer 20 may comprise one or more processors 28, such as a single central processing unit (“CPU”), or a plurality of processing units, commonly referred to as a parallel processing environment, which may execute instructions to practice the various aspects described herein.

Generally, three nominally orthogonal electric fields are generated by a series of driven and sensed electric dipoles (e.g., surface electrode pairs 12/14, 18/19, and 16/22) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any number of electrodes) could be positioned in any other effective arrangement for driving a current to or sensing a current from an electrode in the heart. For example, multiple electrodes could be placed on the back, sides, and/or belly of patient 11. Additionally, such non-orthogonal methodologies add to the flexibility of the system. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17 placed in the heart 10 are exposed to the field from a current pulse and are measured with respect to ground, such as belly patch 21. In practice the catheters within the heart 10 may contain more or fewer electrodes than the sixteen shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which system 8 measures positions. Data sets from each of the surface electrodes, the internal electrodes, and the virtual electrodes may all be used to determine the location of the roving electrodes 17 within heart 10.

The measured voltages may be used by system 8 to determine the location in three-dimensional space of the electrodes inside the heart, such as roving electrodes 17 relative to a reference location, such as reference electrode 31. That is, the voltages measured at reference electrode 31 may be used to define the origin of a coordinate system, while the voltages measured at roving electrodes 17 may be used to express the location of roving electrodes 17 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described, for example, in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

Therefore, in one representative embodiment, system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In some embodiments, system 8 is the EnSite™ Velocity™ or EnSite Precision™ cardiac mapping and visualization system of Abbott Laboratories. Other localization systems, however, may be used in connection with the present teachings, including for example the CARTO navigation and location system of Biosense Webster, Inc., the AURORA® system of Northern Digital Inc., Sterotaxis' NIOBE® Magnetic Navigation System, as well as MediGuide™ Technology from Abbott Laboratories.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

Aspects of the disclosure relate to computing transmurality indices and/or ablation parameters. System 8 can therefore also include a transmurality computation module 58 that can be used to determine transmurality indices and/or to compute ablation parameters from given transmurality indices.

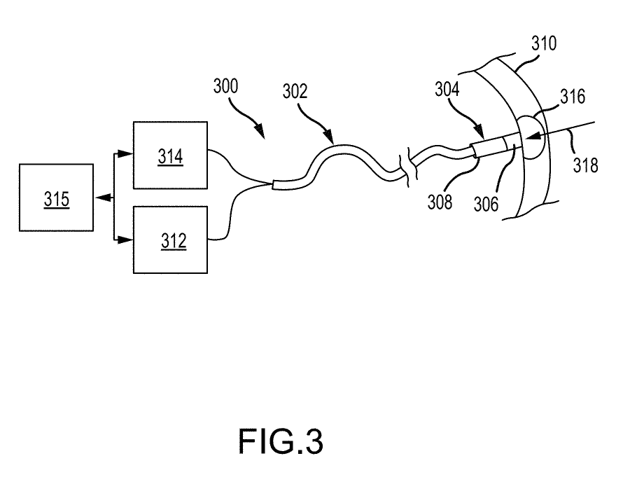

FIG. 3 schematically depicts an exemplary contact ablation system 300. Although illustrated as a standalone system, contact ablation system 300 could also be incorporated into electroanatomical mapping system 8 described above. Contact ablation system 300 includes a catheter 302 having a distal portion 304, which in turn includes an ablation head 306 operatively coupled with a force sensor 308. Ablation head 306 is arranged for contact with a target tissue (that is, a tissue to be ablated) 310.

Catheter 302 is operatively coupled with a power source 312 that provides and measures the energy delivered to ablation head 306. A measurement device 314 is also depicted, capable of sourcing force sensor 308 and measuring an output signal therefrom.

Contact ablation system 300 can also include a central controller 315, such as a computer or microprocessor operatively coupled with power source 312 and measurement device 314 for control thereof and for processing information received therefrom.

In operation, ablation head 306 is brought into contact with target tissue 310 and energized to create a lesion 316 on and within target tissue 310. Force sensor 308 is configured to generate an output from which a magnitude of a contact force vector 318 can be inferred. It should be understood that the contact force can be time-variant, particularly when target tissue 310 is subject to motion (e.g., the wall of a beating heart). The energy flow (e.g., current or power) through ablation head 306 can also be time-variant, as the energy flow may depend on the contact resistance between ablation head 306 and target tissue 310, which in turn can vary with the contact force and the changing properties of lesion 316 during ablation.

U.S. Pat. No. 9,149,327 discloses a lesion size index (“LSI”) related to the contact force F between ablation head 306 and target tissue 310, an energization parameter E applied to target tissue 310 (e.g., power, voltage, and/or current), and the duration time t of the ablation. Each of the F, E, and t parameters is taken into account through an exponential term that models saturation effects. The saturation effect takes into account the asymptotic nature of lesion formation, wherein lesion growth approaches a size limit at infinite time. Also, because the modeling is based on real data, changes in the material properties of the tissue under ablation are accounted for (e.g., changes in the electrical resistivity, which affects the quantity of the heat generated by the joule heating effect).

The effect of these parameters have been modelled and correlated with ablation data from numerous clinical studies to arrive at an equation set based on the model. The LSI can thus be expressed as a retrospective equation or set of equations that can be programmed into central controller 315.

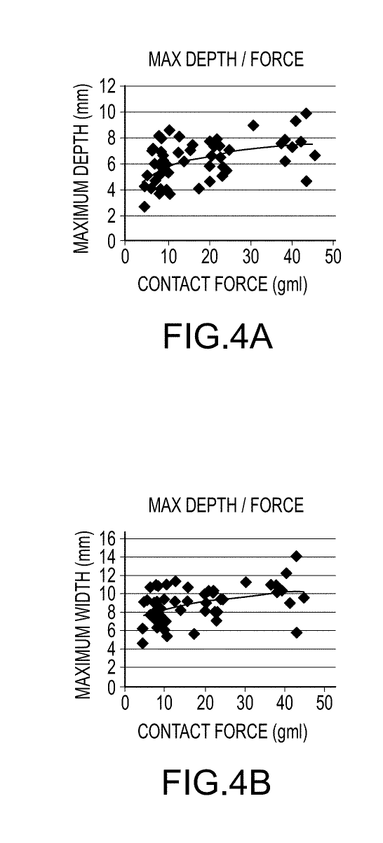

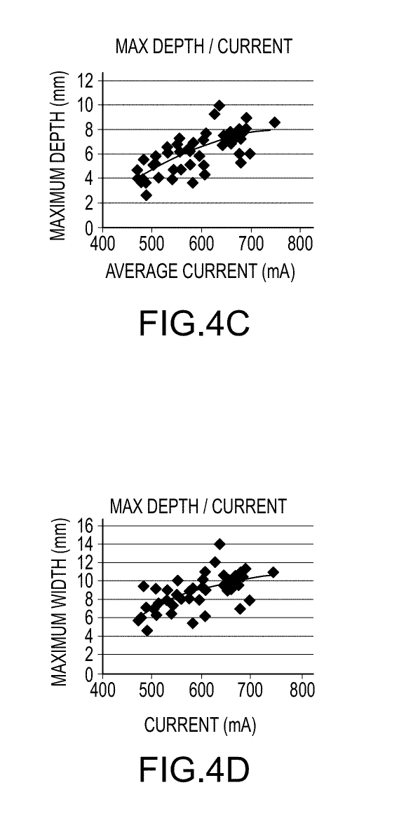

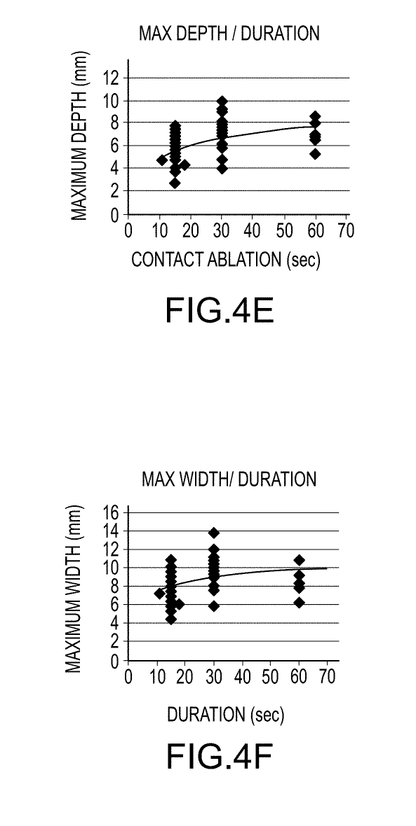

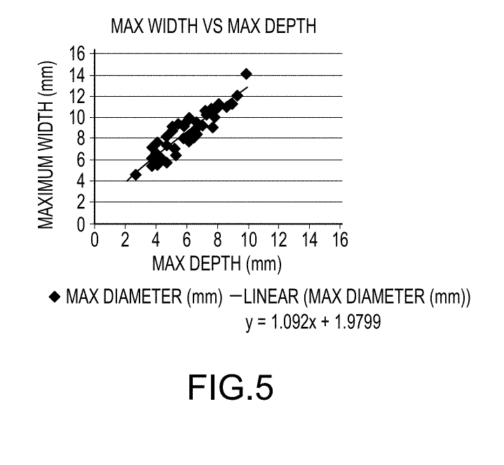

More particularly, and with reference to FIGS. 4A through 4F, data showing the exponential form of the LSI are depicted in an embodiment of the disclosure, demonstrating similar forms of the various lesion width and lesion depth parameters. For these data, the energization parameter E is electrical current. Referring to FIG. 5, the correlation between the lesion width and lesion depth parameters is observable. For the data presented in FIG. 5, a correlation of R=0.91 is realized. The high correlation confirms that the same model can be applied to calculate both the lesion depth index (“LDI”) and the lesion width index (“LWI”).

The retrospective equation that describes the LSI model can be of the general form:

$$LSI ( F , I , t ) = {k}_{1} * ( {f}_{2} ( 1 - {e}^{- \frac{F}{{f}_{1}}} + {f}_{0} ) * {i}_{2} ( 1 - {e}^{- \frac{I}{{i}_{1}}} ) * ( ( 1 - {k}_{0} ) + {k}_{0} \frac{1 - {e}^{- \frac{t}{τ}}}{1 - {e}^{- \frac{T}{τ}}}$$

where f₀, f₁, and f₂ are force parameter coefficients, i₁ and i₂ are electrical current coefficients, k₀ is a diffusive heating coefficient, k₁ is a rescaling coefficient, and τ is a characteristic time value. The input unites for the LSI are grams-force (gmf) for the force F, milliamps (mA) for the current I, and seconds (sec) for the duration time t. The resulting output of the equation above is a length in millimeters (mm).

The LSI model reflected in the equation above includes a joule heating component (1−k₀) that is independent of time and a diffusive heating component

$${k}_{0} \frac{1 - {e}^{- \frac{t}{τ}}}{1 - {e}^{- \frac{T}{τ}}}$$

that is a function of time. The joule heating and diffusive heating components are multiplied by the lesion depth estimated for an ablation lasting a time period of T, with the averaged force F and electrical current I over the time period T. Data analyzed for this work was generated for a time period T of 60 seconds. It is noted that the baseline time of 60 seconds was a result of the availability of lesion data that was based on 60 second ablation times. Data from ablations of different durations (e.g., 30 sec, 45 sec) can also be utilized in a form similar to that given above by substitution of the appropriate time found in the numerator of the diffusive heating component.

The retrospective equation above is a separable variable function of contact force F, electrical current I, and duration time t of the ablation. The parameters of this equation were obtained by best fit of experimental data acquired during preclinical studies. The same general form was utilized to calculate both the LDI and the LWI. Only the best fit coefficients differ between the equations. The various coefficients are presented in Table 1:

TABLE 1
Best Fit Coefficients for LDI and LWI Equations

| | f₂ | f₁ | f₀ | i₂ | i₁ | k₀ | k₁ | T |
|---|---|---|---|---|---|---|---|---|
| LDI | 4.36 | 20.67 | 2.17 | 2.57 | 630.75 | 0.578 | 1.22/√2 | 38.57 |
| LWI | 3.74 | 18.20 | 1.99 | 3.29 | 525.85 | 0.481 | 1.10 | 29.23 |

The k₀ for the LDI includes a separate √2 factor in the denominator for conversion from maximum depth to effective depth. That is, if the LDI of the effective depth is desired, the √2 factor should be included in the calculation.

By implementation of the equation above, the central controller 315 can apprise operators of the estimated lesion growth in essentially real time, as the ablation is in progress.

Development of the LWI is now described. The LWI model considers two aspects of lesion development when computing the lesion width in real time: the completed portion of the growth of the lesion width and the uncompleted portion of the growth of the lesion width, based on a total time T. As explained above, the total time T for this work is 60 seconds because that was the total time of the ablations for the data analyzed for the modelling. Based on observations of the data and the exponential behavior attributed to saturation, the LWI uses the exponential functions of time. The exponential function can be a function of previous time step exponential plus an increment:

$$f ( {t}_{1} ) = A ( 1 - {e}^{- \frac{{t}_{1}}{τ}} ) = f ( {t}_{0} ) + ( A - f ( {t}_{0} ) ) ( 1 - {e}^{- \frac{Δ t}{τ}} ) , Δ t = {t}_{1} - {t}_{0} .$$

Calculations can be gated to be performed only at the time step Δt (e.g., 1 sec) in the interest of computational economy.

In one embodiment, calculations are made with force and current averaged over a migrating averaging window, e.g., over the last n seconds. The migrating averaging window helps account for the phenomenon of thermal latency, as explained in S. K. S. Huang and M. A. Wood, Catheter Ablation of Cardiac Arrhythmia, Chapter 1 (2006). Thermal latency is the mechanism by which the temperature and growth of the lesion continue to rise after energization ceases. Huang and Wood, for example, report that the temperature of the lesion continues to rise for an additional six seconds after cessation of energization. Accordingly, in one embodiment of the disclosure, the time period for the migrating averaging window is 6 seconds.

In part because of the thermal latency effect, the evolution of the lesion is not well known for the first 6 seconds of ablation. Lesions are analyzed post-ablation, and the size of the lesions for short duration ablations is dwarfed by the thermal latency effect. Accordingly, in one embodiment, the LWI is calculated within the first 6 seconds of ablation as a linear interpolation between the origin and the value expected at 6 seconds.

The estimation of what the lesion width would be at time t=T of ablation (LWI_{T}) is the width that the lesion would reach if constant current and force were applied during the whole time period T:

$${LWI}_{T} ( F , I ) = LWI ( F , I , t = T ) = {k}_{1} * ( {f}_{2} ( 1 - {e}^{- \frac{F}{{f}_{1}}} ) + {f}_{0} ) * {i}_{2} ( 1 - {e}^{- {( \frac{I}{{i}_{1}} )}^{2}} ) .$$

The joule heating component of the lesion width index (LWI_{JH}) accounts for the tissue that is heated directly by passage of electrical current applied by the catheter. In one embodiment, LWI_{JH} is thus assumed as the source of heat which then diffuses in the tissue. The LWI_{JH} can also be defined as a constant ratio of the LWI at the total time T (i.e., LWI_{T}):

LWI_{JH}=LWI_{T}(1−k₀).

That is, in one embodiment, the LWI_{JH} component of the lesion formation is constant with respect to time, but is still variable with respect to the energization parameter E and the applied contact force F.

The completed portion of the growth of the lesion width is taken as the LWI at the last time step t0 (LWIₜ₀), or the lesion size due to new joule heating LWI_{JH} if it exceeds the lesion at LWIₜ₀:

max{LWIₜ₀,LWI_{JH}}.

The uncompleted portion of the growth of the lesion is driven by the LWI_{T} and the LWI_{JH}, both using average force and current on the previous 6 seconds.

The actual LWI at time t1 (LWIₜ₁) is the LWIₜ₀ plus an incremental lesion ΔLWI:

$$Δ LWI = ( \frac{{LWI}_{T} * {k}_{0}}{{k}_{3}} - [ max \{ {LWI}_{t 0} , {LWI}_{JH} \} - {LWI}_{JH} ] ) ( 1 - {e}^{- \frac{Δ t}{τ}} ) , \\ Δ t = t 1 - t 0 , {k}_{3} = 1 - {e}^{- \frac{T}{τ}} , {LWI}_{t 1} = max \{ {LWI}_{t 0} , {LWI}_{JH} \} + Δ LWI .$$

Subtracting the LWI_{JH} from the completed portion of the growth of the lesion demonstrates that the exponential characteristics of the LWI and the ΔLWI only applies on the diffusive component.

It is noted that the development of the LDI is the same as the development of the LWI because both indices have the same form and are driven by the same physics. Accordingly, the derivation of LDI is the same as for the LWI, albeit using different data (e.g., depth data).

The lesion volume can be inferred from the lesion width by multiplying a cubic of the maximum width of the lesion by a constant. In one embodiment, the equation for converting from maximum lesion width to lesion volume is given by the equation

Lesion Volume=0.125167*π*[MAX WIDTH]³.

Based on data analyzed for this work, the foregoing equation has a correlation coefficient of R=0.99. Because LWI is based on the maximum width of a lesion, the lesion volume index (“LVI”) is related to the LWI in the same way:

LVI=0.125167*π* LWI³.

The instant disclosure provides methods, apparatuses, and systems to express LSI as a function of tissue biological attributes or properties, such as fiber orientation, tissue thickness, fat (adipose) content, scar content, fibrosis, and the like. This is referred to herein as a “transmurality index” and allows for back-calculation of one or more of ablation energy level (e.g., power, voltage, and/or current), ablation time, and ablation contact force in order to achieve a transmural lesion in tissue of varying biological attributes. That is, according to aspects of the instant disclosure, a transmurality index can be a function of one or more tissue biological attributes, ablation contact force, ablation time, and/or ablation energy level.

For purposes of illustration, transmurality indices will be explained herein in connection with tissue thickness. Those of ordinary skill in the art will appreciate from the instant disclosure how to extend the teachings herein to other tissue biological attributes.

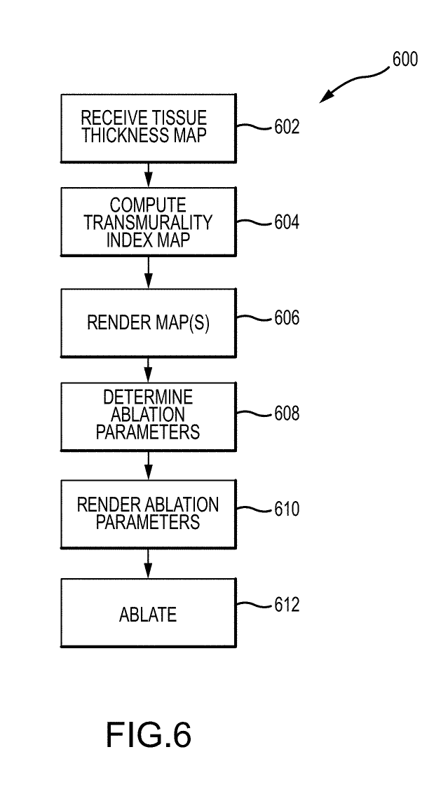

One exemplary method of determining ablation parameters according to the present teachings will be explained with reference to the flowchart 600 of representative steps presented as FIG. 6. In some embodiments, for example, flowchart 600 may represent several exemplary steps that can be carried out by central controller 315 of FIG. 3 and/or by electroanatomical mapping system 8 of FIG. 1 (e.g., by processor 28 and/or transmurality computation module 58). It should be understood that the representative steps described below can be either hardware- or software-implemented. For the sake of explanation, the term “signal processor” is used herein to describe both hardware- and software-based implementations of the teachings herein.

In block 602, a tissue thickness map for a cardiac region to be ablated (e.g., the left ventricle) is received. According to aspects of the disclosure, the tissue thickness map is determined from a segmented model of the cardiac region to be ablated, such as an MRI or CT image of the left ventricle.

In block 604, a transmurality index map is computed from the tissue thickness map. That is, transmurality indices for the target tissue are computed as a function of the thickness of the target tissue.

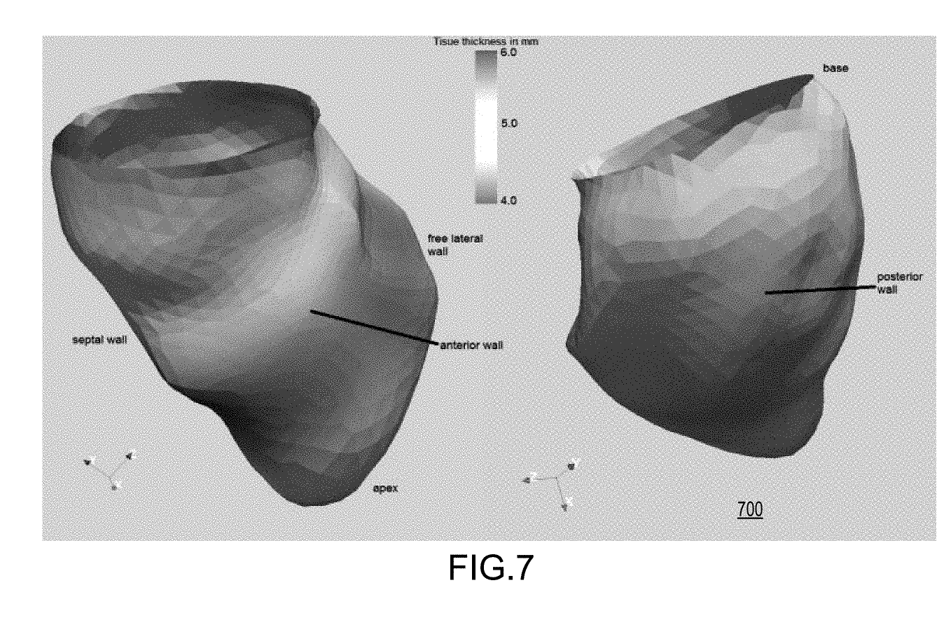

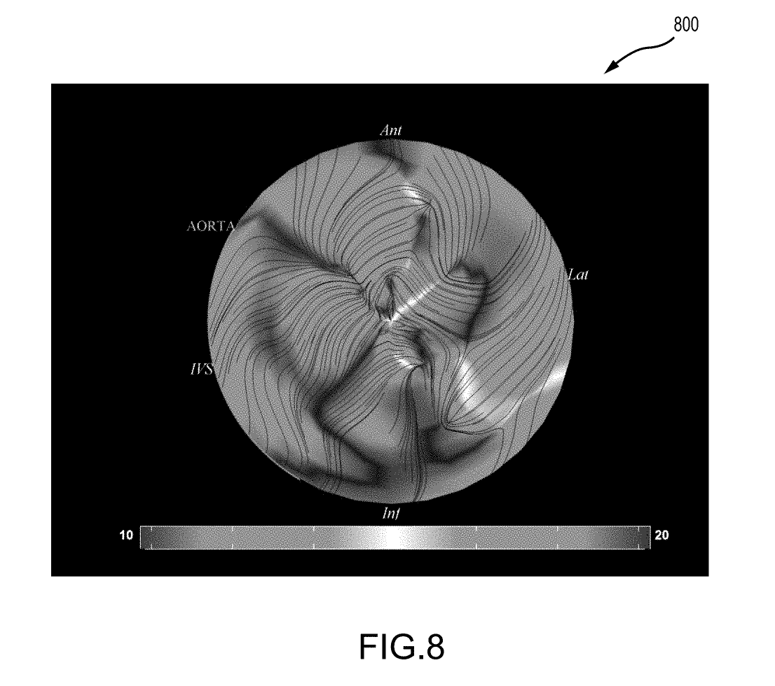

In block 606, one or more of the tissue thickness map and the transmurality index map are rendered graphically. For example, tissue thickness and/or transmurality indices can be output on a geometric model of the cardiac region to be ablated (700, see FIG. 7) or as a bullseye plot (800, see FIG. 8). These graphical renderings provide a practitioner with information regarding the likely transmurality index required at any given point on the target tissue in order to achieve a transmural lesion.

In block 608, one or more ablation parameters (e.g., ablation energy level, ablation time, and ablation contact force) are determined using the transmurality index map. That is, for any given location on the target tissue, the transmurality index computed for that location is used to determine one or more ablation parameters that will likely result in the creation of a transmural lesion at that location.

As those of ordinary skill in the art will appreciate from the discussion of LSI above, the transmurality index can alternatively be expressed as a function of ablation energy level, ablation time, and/or ablation contact force. In fact, just as in the case of the above-described LSI, the effect of these parameters can be modelled and correlated with ablation data from numerous clinical studies in order to express the transmurality index as a retrospective equation or set of equations that can be programmed into central controller 315 and/or electroanatomical mapping system 8 (e.g., processor 28 and/or transmurality module 58).

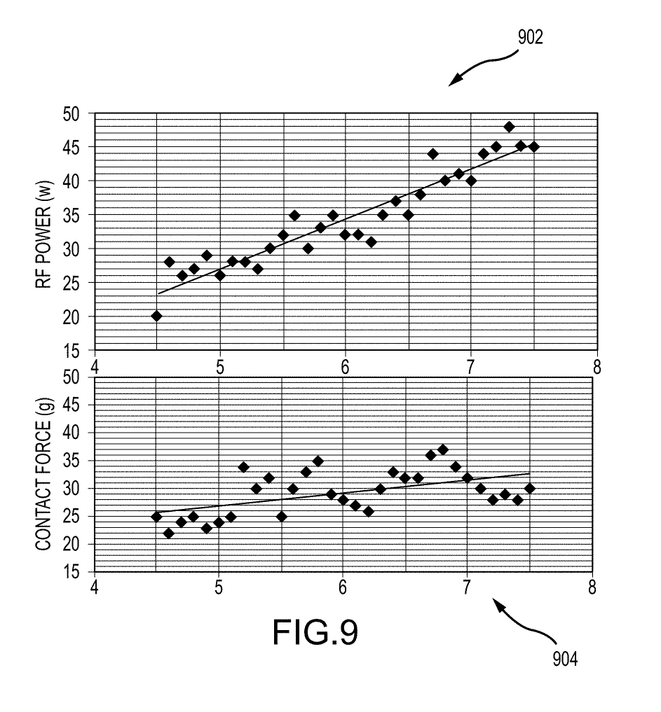

Thus, according to aspects of the instant disclosure, given one or more ablation parameters (e.g., given ablation energy level and ablation contact force), this equation or set of equations can be used to solve for any remaining ablation parameters (e.g., ablation time). In this regard, and by way of example only, FIG. 9 shows representative plots of ablation power 902 and ablation contact force 904 versus transmurality index.

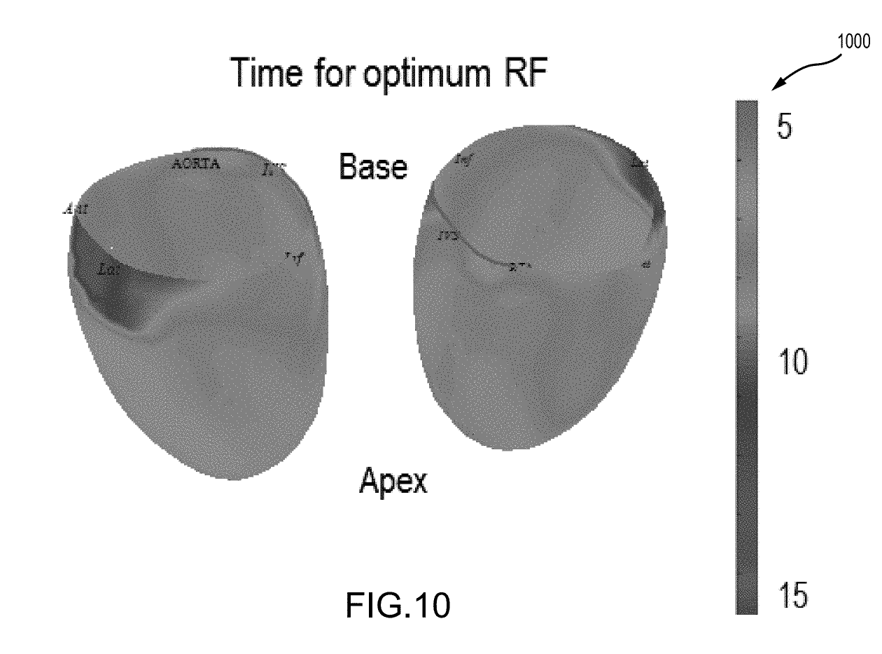

In block 610, one or more ablation parameter(s) can be rendered graphically. For example, numerical value(s) for the ablation parameter(s) can be superimposed upon a three-dimensional cardiac model in order to guide a practitioner in delivering ablation therapy (e.g., by showing the optimal ablation contact force in grams-force, the optimal ablation voltage, current, and/or power, and/or the optimal ablation time for a given location on the target tissue). Alternatively or additionally, the value(s) for the ablation parameter(s) can be expressed in color- or greyscale. FIG. 10 shows a graphical representation 1000 of ablation time expressed in greyscale on a three-dimensional geometric model of the cardiac surface.

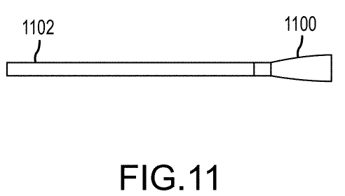

In still other embodiments, the ablation parameter(s) can be output using iconography. For example, as shown in FIG. 11, a cone-shaped icon 1100 can be displayed on a graphical representation 1102 of catheter 302, with the size and shape of icon 1100 varying with the ablation parameter so represented (e.g., the width of icon 1100 can diminish as the required ablation time diminishes). It is also contemplated that similar iconography can be used to graphically represent local tissue thicknesses and/or transmurality indices.

Ablation is carried out according to the ablation parameters in block 612. The determined ablation parameter(s) can be user- and/or automatically-controlled, such as by central controller 315 and/or by electroanatomical mapping system 8 (e.g., processor 28 and/or transmurality module 58).

Although several embodiments have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of determining parameters for cardiac ablation, comprising:
receiving a tissue biological property map for a cardiac region to be ablated;
computing a transmurality index map using the tissue biological property map; and
determining one or more of ablation energy level, ablation time, and ablation contact force to achieve a transmural lesion using the computed transmurality index map.

2. The method according to claim 1, further comprising outputting a graphical representation of the tissue biological property map.

3. The method according to claim 1, wherein the tissue biological property map comprises a tissue thickness map.

4. The method according to claim 3, further comprising outputting an iconographic indication of local tissue thickness on a geometric model of an ablation catheter.

5. The method according to claim 1, further comprising outputting a graphical representation of the transmurality index map.

6. The method according to claim 5, wherein outputting a graphical representation of the transmurality index map comprises outputting the graphical representation of the transmurality index map on a geometric model of the cardiac region to be ablated.

7. The method according to claim 5, wherein outputting a graphical representation of the transmurality index map comprises outputting the graphical representation of the transmurality index map as a bullseye plot.

8. The method according to claim 1, further comprising graphically outputting the one or more of ablation energy level, ablation time, and ablation contact force.

9. The method according to claim 8, wherein graphically outputting the one or more of ablation energy level, ablation time, and ablation contact force comprises displaying a numerical value for the one or more of ablation energy level, ablation time, and ablation contact force on a geometric model of the cardiac region to be ablated.

10. The method according to claim 1, wherein receiving a tissue biological property map for a cardiac region to be ablated comprises:
receiving a segmented model of the cardiac region to be ablated; and
determining the tissue biological property map from the segmented model.

11. The method according to claim 1, wherein determining one or more of ablation energy level, ablation time, and ablation contact force to achieve a transmural lesion using the computed transmurality index map comprises, given values for two of ablation energy level, ablation time, and ablation contact force, determining a remaining one of ablation energy level, ablation time, and ablation contact force from the computed transmurality index map.

12. A method of performing cardiac ablation, comprising:
computing a transmurality index map using tissue thickness information for a cardiac region to be ablated;
determining one or more of ablation energy level, ablation time, and ablation contact force to achieve a transmural lesion within the cardiac region to be ablated from the transmurality index map; and
delivering ablation energy to the cardiac region to be ablated according to the determined one or more of ablation energy level, ablation time, and ablation contact force.

13. The method according to claim 12, wherein computing a transmurality index map using tissue thickness information for a cardiac region to be ablated comprises computing a transmurality index map using tissue thickness information derived from a segmented model of the cardiac region to be ablated.

14. The method according to claim 12, further comprising outputting a graphical representation of the transmurality map on at least one of a bullseye plot and a geometric model of the cardiac region to be ablated.

15. The method according to claim 12, wherein determining one or more of ablation energy level, ablation time, and ablation contact force to achieve a transmural lesion within the cardiac region to be ablated from the transmurality index map comprises, given values for two of ablation energy level, ablation time, and ablation contact force, determining a remaining one of ablation energy level, ablation time, and ablation contact force using the transmurality index map.

16. The method according to claim 12, further comprising graphically outputting the one or more of ablation energy level, ablation time, and ablation contact force.

17. The method according to claim 16, wherein graphically outputting the one or more of ablation energy level, ablation time, and ablation contact force comprises displaying a numerical value for the one or more of ablation energy level, ablation time, and ablation contact force on a geometric model of the cardiac region to be ablated.

18. The method according to claim 12, further comprising graphically outputting the tissue thickness information for the cardiac region to be ablated as iconography on a geometric model of an ablation catheter.

19. A cardiac ablation control system, comprising:
an ablation parameter determination processor configured to:
receive as input a tissue thickness map for a cardiac region to be ablated;
compute a transmurality index map using the tissue thickness map; and
determine one or more ablation parameters selected from the group consisting of ablation energy level, ablation time, and ablation contact force to achieve a transmural lesion in the cardiac region to be ablated from the computed transmurality index map.

20. The system according to claim 19, wherein the ablation parameter determination processor is further configured to:
receive as input values for two ablation parameters; and
compute a value for a remaining ablation parameters from the values received as input for the two ablation parameters and the computed transmurality index map.

21. The system according to claim 19, wherein the ablation parameter determination processor is further configured to graphically output the determined one or more ablation parameters.
